(19) United States
(12) Patent Application Publication
(10) Pub. No.: US 2017/0216375 A1
(43) Pub. Date: Aug. 3, 2017

(54) Neonatal Microbiome Supplementation

(71) Applicant: BIOGAIA AB, Stockholm (SE)

(72) Inventors: Bo MÖLLSTAM, Lerum (SE); Kjersti AAGAARD, Bellaire, TX (US)

(21) Appl. No.: 15/312,465

(22) PCT Filed: May 20, 2015
(86) PCT No.: PCT/EP2015/061181

§ 371 Date: Nov. 18, 2016

Related U.S. Application Data
(60) Provisional application No. 62/000,842, filed on May 20, 2014.

(51) Int. Cl. A61K 35/747; A61K 35/745

(52) U.S. Cl. CPC A61K 35/747; A61K 35/745

(57) ABSTRACT

The present invention aims to prevent and/or treat health-related conditions in infants born with a non-optimal intestinal microbiome, by providing specific probiotic lactic acid bacterial strains to the pregnant mother. This will enable early colonization of the infant gut, even before birth, and thus prevent and/or treat health-related conditions associated with non-optimal microbiome. Thus, the present invention provides a probiotic microorganism for use in the treatment of a fetus or neonate, wherein said probiotic microorganism is administered to the oral cavity of a pregnant female in a formulation suitable to colonize the oral cavity, wherein said probiotic microorganism is thereby administered to the fetus. The methods and uses of the invention can also provide for the colonization of a fetus or neonate or for shifting the microbiome of a fetus or neonate

### FIELD OF THE INVENTION

The present invention generally relates to the neonatal microbiome. More specifically the invention relates to methods for supplementation of the neonatal microbiome. This invention relates to an early colonization of probiotic bacteria of the neonatal microbiome and thus a chance for an improved life outside the uterus. Such early colonization will prevent and/or treat health related diseases or conditions associated with infants born with a non-optimal intestinal flora.

### BACKGROUND OF THE INVENTION

The human microbiome is the total collection of all microorganisms (such as bacteria, fungi, and viruses) that resides on the surface and in deep layers of skin, in the nasal and oral cavities, in the gastrointestinal tracts, in the urogenital systems and in other locations of the human body. Bacterial cells were historically often considered only as harmful due to their ability to cause infection. However, there are certain beneficial characteristics associated with some bacteria. They are often referred to as probiotic bacteria. The word “probiotic” stems from the Greek word pro, which means “promoting” and biotic, which means “life”. The Food and Agricultural Organization of the United Nations define probiotics as “live microorganisms which when administered in adequate amounts confer a health benefit on the host”. Certain strains of lactic-acid producing bacteria such as Lactobacillus and Bifidobacteria are commonly used as probiotics in various types of foods, for example yoghurt. Growth and colonization of harmful microorganisms can be prevented by lactic acid producing bacteria through their own colonization on or inside the mammal, through formation of bio films, through competition of available nutrients and also the production of specific substances such as hydrogen peroxides, bacteriocines, organic acids (including lactic acid and acetic acid) that lower the pH. A mammal can benefit from probiotic bacteria through a plethora of ways and the effectiveness of the probiotic bacteria is strain-specific, where each strain may contribute to mammal health through different mechanisms. Probiotics can prevent or inhibit the proliferation of pathogens, suppress production of virulence factors by pathogens, or modulate the immune response in a pro-inflammatory or an anti-inflammatory way.

Newborn babies have a complex microbiome that resides in the gut already within the first week after birth, but it takes up to 3 years until the bacterial composition has matured and reached some kind of equilibrium. Premature infants often show a different microbe colonization of the gut from start and they are often found to have intestinal problems during the first weeks of life. Necrotizing enterocolitis (NEC) is a medical condition primarily seen in premature infants. The condition is associated with necrosis of parts of the bowel. The symptoms appear after birth and in worst case it can lead to death. NEC is recognized as the second most common cause of morbidity in premature infants in the United States and sadly there is no effective cure or treatment for NEC today.

Infant colic, or baby colic, is a common and often frustrating problem for parents and caregivers. The condition is one of the most regular reasons for visits and contacts with pediatricians, family practitioners and community nurses. Infant colic can be referred to as a clinical condition of inconsolable crying, fussing and irritability in at least three hours in more than three weeks during the first three months of life. It appears most often in the evenings, in an otherwise healthy baby. The exact cause of infant colic remains yet unknown, but there are most likely several factors associated with the outbreak of the condition. In recent years the role of the microbiome that resides in the infant gut has been discussed as one possibility. A shift in bacterial load has been observed due to lower amounts of intestinal lactobacilli compared to higher amounts of coliform bacteria in a comparison between colicky infants and healthy ones.

Early colonization of the gastrointestinal tracts is essential in order for the immune system to develop properly during the first years of life. The most widespread understanding is that the fetus intestine is sterile and that the newborn is inoculated with the mother's vaginal and rectal bacterial flora during natural birth. Children delivered by caesarean section have a less diverse bacterial flora during its first two years of life compared to children born in a natural way. The risk for developing allergy is higher for children delivered by caesarean section.

Thus, there is a present need for new and better treatments for ensuring a well-developed microbiome in the infant gut.

### SUMMARY OF THE INVENTION

The present invention aims to solve (e.g. prevent and/or treat) health-related conditions associated with or found in infants born with a non-optimal intestinal flora or microbiome, by providing specific probiotic lactic acid bacterial strains to the pregnant mother. This will enable early colonization of the infant gut, even before birth and thus prevent and/or treat health-related conditions associated with non-optimal microbiome in infants or neonates.

The present invention provides the use of certain probiotic bacteria, specifically the use of lactic acid probiotic bacteria for their specific characteristics, for prophylactic treatment to prevent certain diseases and conditions, for the benefit of the infant.

More specifically the invention relates to administering probiotic bacteria, specifically lactic acid bacteria, to the pregnant mother, and having the bacteria colonize the infant early in life. Early colonization of specific probiotic bacteria is beneficial for the infant.

A primary object of the present invention is to prepare the fetus for a life outside the uterus, for example to prevent and/or treat health-related conditions associated with non-optimal intestinal flora or microbiome.

An object is to administer certain probiotic bacteria orally to the pregnant mother, for subsequent colonization of its offspring, e.g. the neonate or infant which develops from the fetus carried in the uterus of the pregnant mother.

Another object is to allow the probiotic bacteria to colonize the fetus gut by hematogenous spread from the mother's oral cavity.

Another object is to allow the probiotic bacteria to shift the microbiome of the mothers oral cavity and then this shift is reflected in the microbiome of the fetus gut by hematogenous spread from the mother's oral cavity.

A further object is to administer certain probiotic bacteria orally to the mother, in the form of chewable tablets, chewing gums or lozenges or the like to maximize the exposure time in the oral cavity of the mother and thus the successful colonization of at least parts of the mother's oral cavity and consequently the gut of the fetus.

Another object is to administer certain probiotic bacteria orally to the mother, in the form of chewable tablets, chewing gums or lozenges or the like to maximize the exposure time in the oral cavity of the mother and thus the successful colonization of at least the tonsils of the mother's oral cavity and consequently the gut of the fetus.

Another object is to administer certain probiotic bacteria orally to the mother, in the form of chewable tablets, chewing gums or lozenges or the like to maximize the exposure time in the oral cavity of the mother and thus the successful colonization of for example at least the tooth surface of the mother's oral cavity and consequently the gut of the fetus.

Another object is to administer certain probiotic bacteria orally to the mother, in the form of chewable tablets, chewing gums or lozenges or the like to maximize the exposure time in the oral cavity of the mother and thus the successful colonization of at least the gingival sulcus of the mother's oral cavity and consequently the gut of the fetus.

An object of the invention is to supplement the gastrointestinal microbiome in an infant with certain probiotic bacteria, specifically certain lactic acid bacteria, to ensure a microbiome comprising a beneficial composition of bacteria.

Another object is the use of certain probiotic bacteria, specifically the use of certain lactic acid probiotic bacteria known for their capability of prophylactic treatment of colic for the benefit of the infant in order to minimize the risk for developing infant colic.

Another object is the use of certain probiotic bacteria, specifically the use of certain lactic acid probiotic bacteria for their anti-inflammatory characteristics, for prophylactic treatment for the benefit of the infant in order to minimize the risk for developing NEC.

Another object is the use of certain probiotic bacteria, specifically the use of certain lactic acid probiotic bacteria known for their capability of prophylactic treatment for reducing allergies for the benefit of the infant in order to minimize the risk for immune associated conditions such as allergy.

Another object is the use of certain probiotic bacteria, specifically the use of certain lactic acid bacteria for prophylactic treatment of the mother to minimize the risk of preterm delivery.

Beneficial probiotics should be administered to the mother's oral cavity before and/or during pregnancy.

Beneficial probiotics, such as certain lactic acid bacteria, should be administered to the mother's oral cavity before and/or during pregnancy.

The present invention is based on the surprising finding that probiotic microorganisms present or administered to the oral cavity of a pregnant female can in turn be administered or transferred to the fetus and then to the subsequent neonate and infant. Whilst not wishing to be bound by theory, it is believed that administration of the probiotic microorganisms to the oral cavity of a pregnant female such that at least part of the oral cavity is colonized by said probiotic bacteria then enables the hematogenous spread of the probiotic microorganism from the mother's oral cavity to the placenta and the fetus and thereby to colonize the fetus gut/intestine. Indeed, the attached Example demonstrates that certain probiotic bacteria can be administered to pregnant animals and that said bacteria are then detected in the placenta and in the new born pups.

Thus, the present invention provides a probiotic microorganism for use in the treatment of a fetus or neonate (e.g. the treatment or prevention of a disease or condition in a fetus or neonate), wherein said probiotic microorganism is administered to the oral cavity of a pregnant female in a formulation suitable to colonize the oral cavity, wherein said probiotic microorganism is thereby administered to the fetus.

Alternatively viewed, the present invention provides a probiotic microorganism for use in the administration of said probiotic microorganism to a fetus or neonate, wherein said probiotic microorganism is administered to the oral cavity of a pregnant female in a formulation suitable to colonize the oral cavity.

Thus, the probiotic microorganisms are administered to a fetus in-utero by administration of the microorganisms to the mother.

In addition the present invention provides a probiotic microorganism for use in the colonization of a fetus or neonate, wherein said probiotic microorganism is administered to the oral cavity of a pregnant female in a formulation suitable to colonize the oral cavity, wherein said probiotic microorganism thereby colonizes the fetus.

Alternatively viewed, the present invention provides a probiotic microorganism for use in the colonization of a fetus or neonate with said probiotic microorganism, wherein said probiotic microorganism is administered to the oral cavity of a pregnant female in a formulation suitable to colonize the oral cavity.

Preferably, the present invention enables early colonization of the fetus or neonate, in particular the gut or intestine of the fetus or the neonate which develops from the fetus.

In other aspects the methods or uses of the invention can be used to allow the probiotic microorganism to shift the microbiome of a fetus or neonate, for example in the gut or intestine of the fetus or the neonate which develops from the fetus, by shifting the microbiome of the oral cavity of the mother. Thus, the present invention also provides a probiotic microorganism for use in shifting the microbiome of a fetus or neonate, wherein said probiotic microorganism is administered to the oral cavity of a pregnant female in a formulation suitable to colonize the oral cavity, wherein said probiotic microorganism is thereby administered to the fetus and results in a shift in the microbiome of the fetus.

In an alternative aspect, the methods or uses of the invention can also be used for the supplementation of a fetal or neonatal microbiome, for example the gut or intestinal microbiome of a fetus or the neonate which develops from the fetus, with probiotic microorganisms, for example to ensure a microbiome comprising a beneficial composition of bacteria. Thus, the present invention also provides a probiotic microorganism for use in supplementing the microbiome of a fetus or neonate, wherein said probiotic microorganism is administered to the oral cavity of a pregnant female in a formulation suitable to colonize the oral cavity, wherein said probiotic microorganism is thereby administered to the fetus and results in a supplementation of the microbiome of the fetus.

The present invention also provides the use of a probiotic microorganism in the manufacture of a composition or medicament for use in the treatment of a fetus or neonate (e.g. the treatment or prevention of a disease or condition in a fetus or neonate), wherein said probiotic microorganism is administered to the oral cavity of a pregnant female in a formulation suitable to colonize the oral cavity, wherein said probiotic microorganism is thereby administered to the fetus.

Alternatively viewed, the present invention provides the use of a probiotic microorganism in the manufacture of a composition or medicament for use in the administration of said probiotic microorganism to a fetus or neonate, wherein said probiotic microorganism is administered to the oral cavity of a pregnant female in a formulation suitable to colonize the oral cavity.

Thus, the probiotic microorganisms are administered to a fetus in-utero by administration of the microorganisms to the mother.

In addition the present invention provides the use of a probiotic microorganism in the manufacture of a composition or medicament for use in the colonization of a fetus or neonate, wherein said probiotic microorganism is administered to the oral cavity of a pregnant female in a formulation suitable to colonize the oral cavity, wherein said probiotic microorganism thereby colonizes the fetus.

Alternatively viewed, the present invention provides the use of a probiotic microorganism in the manufacture of a composition or medicament for use in the colonization of a fetus or neonate with said probiotic microorganism, wherein said probiotic microorganism is administered to the oral cavity of a pregnant female in a formulation suitable to colonize the oral cavity.

Preferably, the present invention enables early colonization of the fetus or neonate, in particular the gut or intestine of the fetus or the neonate which develops from the fetus.

In other aspects the methods or uses of the invention can be used to allow the probiotic microorganism to shift the microbiome of a fetus or neonate, for example in the gut or intestine of the fetus or the neonate which develops from the fetus, by shifting the microbiome of the oral cavity of the mother. Thus, the present invention also provides the use of a probiotic microorganism in the manufacture of a composition or medicament for use in shifting the microbiome of a fetus or neonate, wherein said probiotic microorganism is administered to the oral cavity of a pregnant female in a formulation suitable to colonize the oral cavity, wherein said probiotic microorganism is thereby administered to the fetus and results in a shift in the microbiome of the fetus.

In an alternative aspect, the methods or uses of the invention can also be used for the supplementation of a fetal or neonatal microbiome, for example the gut or intestinal microbiome of a fetus or the neonate which develops from the fetus, with probiotic microorganisms, for example to ensure a microbiome comprising a beneficial composition of bacteria. Thus, the present invention also provides the use of a probiotic microorganism in the manufacture of a composition or medicament for use in supplementing the microbiome of a fetus or neonate, wherein said probiotic microorganism is administered to the oral cavity of a pregnant female in a formulation suitable to colonize the oral cavity, wherein said probiotic microorganism is thereby administered to the fetus and results in a supplementation of the microbiome of the fetus.

Further aspects of the present invention provide a method of treatment of a fetus or neonate with a probiotic microorganism, comprising the administration of said probiotic microorganism to the oral cavity of a pregnant female in an amount of oral formulation suitable to (or effective to) colonize the oral cavity of said pregnant female, wherein said probiotic microorganism is thereby administered to the fetus.

Alternatively viewed, the present invention provides a method for the administration of a probiotic microorganism to a fetus or neonate, comprising the administration of said probiotic microorganism to the oral cavity of a pregnant female in an amount of oral formulation suitable to (or effective to) colonize the oral cavity of said pregnant female, wherein said probiotic microorganism is thereby administered to the fetus.

Another aspect of the invention provides a method for colonization of a fetus or neonate with a probiotic microorganism, comprising the administration of said probiotic microorganism to the oral cavity of a pregnant female in an amount of oral formulation suitable to (or effective to) colonize the oral cavity of said pregnant female, wherein said probiotic microorganism thereby colonizes the fetus.

Another aspect of the invention provides a method for shifting the microbiome of a fetus or neonate, comprising the administration of a probiotic microorganism to the oral cavity of a pregnant female in an amount of oral formulation suitable to (or effective to) colonize the oral cavity of said pregnant female, wherein said probiotic microorganism is thereby administered to the fetus and results in a shift in the microbiome of the fetus.

Another aspect of the invention provides a method for supplementing the microbiome of a fetus or neonate, comprising the administration of a probiotic microorganism to the oral cavity of a pregnant female in an amount of oral formulation suitable to (or effective to) colonize the oral cavity of said pregnant female, wherein said probiotic microorganism is thereby administered to the fetus and results in a supplementation of the microbiome of the fetus.

### DESCRIPTION OF FIGURES

FIG. 1: Schematic of planned human clinical study.

FIG. 2: Sampling schedule of planned human clinical study.

### DETAILED DESCRIPTION OF THE INVENTION AND PREFERRED EMBODIMENTS THEREOF

To facilitate understanding of the invention, a number of terms are defined below.

“Fetus” refers to an unborn child when it is still in the mother's uterus.

“Neonatal”, or “neonate” and “newborn” are used interchangeably and refer to a newborn child and the time period directly after birth.

“Infant” and “baby” are used interchangeably and refer to a newborn baby and its first year of life.

“Preterm” refers to a birth that takes place before week 37 of pregnancy.

“Administering”, or “administration” a probiotic bacterium to a subject refers to the oral delivery of said probiotic bacteria to the subject.

“Infection” refers to the invasion of a disease-causing bacterium.

“Clinical condition” refers to a disease or illness that causes observable symptoms.

During pregnancy, the gravidae is subject to numerous anatomical, physiological, and biochemical changes. It is a result of hormonal and physical alterations, and influences the whole body and its organs. The human microbiome is no exception and both the gut and the vagina have been heavily studied to date. The microbiome is expected to play a role in timing of parturition and notably preterm birth. The common understanding today is that preterm birth is initiated by an ascending infection from the vaginal cavity to the placenta. Although there is evidence that there are bacteria present in the amniotic fluid and placenta in pre-term patients as well as in newborns generally, the inventors have surprisingly found out that these bacteria appear to be similar with bacteria normally found in the oral cavity. This later finding therefore implies that the mother's oral microbiota can be transferred to the infant.

Surprisingly, the inventors come up with the idea to administer certain probiotic bacteria to the mother and let them colonize the oral cavity in order to possibly be spread via the blood to the fetus, and thus inoculating the neonatal gut with beneficial probiotic bacteria that properly prepares the neonate for a normal development.

The duration of the intake or administration of the probiotics may vary. The expectant mother may start to take the probiotics as soon as she is aware of her pregnancy. However, the administration may also start before pregnancy. If the expecting mother is in a special risk group for preterm delivery, the administration may start as soon as the pregnancy is known. Administration may start relatively late in the pregnancy, for example at month 3, 4, 5, 6, 7, 8, 9 of pregnancy. Thus, administration can take place throughout pregnancy or late in the pregnancy, for example in the last few weeks (for example up to the last 12 weeks, e.g. the last 1, 2, 3, 4, 5, 6, 7, 8, 9, 10, 11 or 12 weeks) of the pregnancy. For a human pregnancy, administration can for example take place at gestation weeks 28 to 36, or weeks 28 to the end of the pregnancy. Appropriate equivalent timescales can be determined for other mammals depending on the length of gestation.

The oral cavity is made up of different surfaces and areas where bacteria can reside. The different sites of the oral cavity represent their own microflora, and some species are more site-specific whereas others are more commonly found in several areas. One way of dividing the oral cavity into subsites is: dorsum of the tongue, lateral sides of the tongue, buccal fold, hard palate, soft palate, labial gingiva, tonsils of soft tissue surfaces, supragingival, gingival sulcus and subgingival plaques from tooth surfaces. The tonsils appear to be the site containing the highest number of different bacterial species, followed by the tooth surface, the subgingival, and the hard and soft palate. The invention disclosed herein preferably refers to probiotic bacteria that would colonize the parts of the mouth of the mother, which are most associated with hematogenous spread to the placenta.

Neonates have a complex microbiome that resides in the gut already within the first week after birth, but it is not until around the age of 1 to 3 years that the bacterial composition have reached a relatively developed equilibrium. All factors that shape these earliest microbial communities, and at what point the neonate is first exposed to and colonized by its microbiome, remain unclear. In the first week of life, the neonatal gut microbiome is dominated by colonization of certain bacteria namely Actinobacter (mostly Bifidobacterium), Proteobacteria, Bacteroides, and a minor part consists of Firmicutes (including the Lactobacillus spp, normally dominating the vaginal flora). In contrast, the gut microbiome of neonates with low birth weight shows a different composition of bacteria. Their microbiome is dominated by both Firmicutes and Tenericutes phyla, with much less Actinobacter.

Necrotizing enterocolitis (NEC) is an inflammatory disease of the gut that affects preterm infants. The disease leads to an inflammation of parts of the gut that eventually start to die. Common symptoms of the disease include problems with feeding, abdominal distension and bloody stools. If untreated, the disease can cause peritonitis and sepsis due to leakage of gut contents into the abdomen initiated by a perforation of the gut. NEC is considered to be a life-threatening disease. The exact cause is not totally understood but a common opinion is that the infants gut and immune system have not developed properly, especially the intestinal flora is known to be altered.

Infant colic, or baby colic, is a common and often frustrating problem for parents and caregivers. The causes of infant colic remains yet unknown, but most likely several factors are associated with the outbreak of the condition. In recent years the role of the microbiome that resides in the infant gut has been discussed as one possibility. In a multicentre, double-blind, placebo-controlled study it was investigated whether Lactobacillus reuteri DSM 17938 could reduce the onset of colic, constipation and regurgitation in term newborns. The results came out positive and also showed that infants in the Lactobacillus reuteri DSM 17938 group had an improved gut motility compared to the infants in the placebo group. This investigation concludes that prophylactic use of Lactobacillus reuteri DSM 17938 reduced the onset of functional gastrointestinal disorders. This further implies the benefits of using the strain Lactobacillus reuteri DSM 17938 as a prophylactic treatment before birth by orally administration of the probiotic bacteria to the mother.

Thus, it can be seen from the above that in the methods and uses of the present invention, the primary subject or patient to be treated or affected by the methods or uses is the fetus of the pregnant female or pregnant mother, and thereby the neonate or infant which develops from the fetus. However, the administration of the probiotic microorganism to the fetus takes place via the mother. The mother or pregnant female can of course also benefit from the methods and uses of the invention. Thus, the mother or pregnant female is also a subject or patient (e.g. a secondary subject or patient) to be treated or affected by the methods and uses of the invention. The methods or uses of the invention are generally carried out on a patient or subject in need thereof and said methods or uses optionally involve a step in which patients or subjects in need of treatment are identified in any appropriate way, e.g. by looking for or detecting biomarkers or other indicators of the particular disease or condition.

The methods or uses of the invention can be carried out on any pregnant female or indeed on any female before pregnancy or a female who is planning to get pregnant or in the process of trying to get pregnant. Thus, the methods or uses of the invention are generally appropriate for mammals of a child or young/pup bearing age, e.g. in humans a woman of less than 55, 50 or 45 years of age. As the methods or uses of the invention also allow for the treatment of the female subjects, particularly preferred females, e.g. pregnant females, are those suffering from diseases which can be treated or prevented by probiotic microorganisms, for example females with or at risk of developing gingivitis or urinary tract infections or women at risk of premature or pre-term birth and/or having babies with low birth weight.

The methods and uses of the invention can be carried out on any mammal, for example humans or any livestock, domestic or laboratory animal. Specific examples include mice, rats, pigs, cats, dogs, sheep, rabbits, cows and monkey. Preferably, however, the mammal is a human.

The methods and uses of the present invention involve the oral cavity of the female to be colonized with the probiotic microorganism which it is desired to administer or transfer to the fetus. The terms “colonize the oral cavity” or “oral colonization” or other equivalent terms as used herein are given their standard and art understood meaning of the presence and preferably multiplication of a probiotic microorganism in the oral cavity. The reference to multiplication means that the microorganisms are growing or reproducing. Thus, this term encompasses the detectable presence or existence of a probiotic microorganism in at least part of the oral cavity, i.e. at least part of the oral cavity is colonized by the probiotic microorganism.

Thus, for example, the present invention involves a probiotic microorganism being present in the oral cavity of a female for a reasonable or appropriate period of time, and preferably to allow contact between the microorganism and the surface of the oral cavity such that the microorganism can establish a detectable presence or to colonize the oral cavity so as to preferably allow multiplication of the microorganism within the oral cavity. In other words, the presence of the probiotic microorganism in the oral cavity is not transitory, for example the presence of the microorganism can still be detected in the oral cavity after the time window that the microorganism is being administered. This is in contrast for example to a situation in which a probiotic microorganism is given as a standard coated tablet or capsule, e.g. a gelatin coated capsule, which is swallowed, in which case the microorganism has only a transitory existence in the oral cavity during the passage of the tablet into the gastrointestinal tract, and the microorganism is not present in the oral cavity in an appropriate location to colonize the oral cavity.

Thus, the appropriate administration routes, formats and formulations to be used in the present invention are such that contact between the microorganisms and the gastrointestinal tract is not required. Indeed, such contact is not desirable. What is important is that the probiotic microorganism to be administered or transferred to the fetus in accordance with the present invention is in contact with the oral cavity of the female for a sufficient length of time and/or at an appropriate location in the oral cavity to result in oral colonization or to allow oral colonization to occur.

Thus, in the methods and uses of the present invention, the probiotic microorganism is administered in a format or formulation such that the oral cavity of the female is colonized with the microorganism, for example in a format or formulation which will allow direct contact between the microorganism and the oral cavity, for example the surface of the oral cavity, for an appropriate amount of time and/or in an appropriate location to enable colonization of the oral cavity (or at least part of the oral cavity) by the microorganism. Thus, the probiotic microorganisms of the invention are not generally administered in the form of coated tablets or protected capsules or other formats or formulations which are designed to release their microorganism content and have contact with surfaces of the body in the gastrointestinal tract, for example the lower gastrointestinal tract.

Preferred formats and formulations for use in the present invention are thus chewable or suckable formats and formulations, for example chewing gums or chewable tablets or suckable tablets such as lozenges, or some other format or formulation which will remain in the oral cavity or mouth of the female long enough to enable colonization of the oral cavity (or at least part of the oral cavity) with the microorganisms to eventually take place. Alternatively, thick syrups which for example allow the oral cavity to be coated for a prolonged period of time might be used.

Appropriate methods of determining whether or not a probiotic microorganism has colonized the oral cavity or at least part of the oral cavity would be standard and well known to a person skilled in the art and any of these can be used. Such methods can also be used for example to determine the appropriateness of a probiotic microorganism for use in the methods and uses of the present invention. For example, samples can be taken from the oral cavity, for example one or more locations in the oral cavity, and tested for the presence and amount of particular microorganisms. Exemplary locations are given elsewhere herein, for example the hard palate, gingiva (e.g. gingival sulcus), and buccal areas. Other exemplary locations are the tonsils and the tooth surface.

Preferably the microorganism would be detected in the oral cavity if an appropriate sample was to be taken after administration of the probiotic microorganism to the oral cavity in accordance with the present invention.

As described elsewhere herein in accordance with the present invention colonization of the oral cavity of the female then gives rise to transfer of the probiotic microorganism to the fetus or administration (indirect administration) of the probiotic microorganism to the fetus and hence to the neonate and infant. Thus, the colonization of the oral cavity by the probiotic microorganism should be at a level and location within the oral cavity to allow such transfer or administration from the oral cavity to the fetus to occur. In turn, the level of transfer or administration of the probiotic microorganism to the fetus is preferably such that colonization of the fetus, e.g. the fetus gut, occurs, thereby allowing a physiological or therapeutic effect to be observed in the fetus, neonate or infant.

As the present invention allows the transferal of probiotic microorganisms from the mother to the fetus in-utero, it can be seen that the present invention can be used to treat or prevent any disease or condition in a fetus, neonate or infant which can benefit from treatment with probiotic microorganisms. Exemplary conditions for treatment will thus already have been recognised. The present invention can also be used for the treatment or prevention of a health-related disease or condition associated with neonates or infants born with a non-optimal intestinal flora or microbiome. Thus, examples of diseases or conditions which can be treated or prevented in accordance with the present invention include intestinal problems or gastrointestinal disorders, allergy or other immune associated conditions, inflammatory conditions (for example inflammatory diseases of the gut), intestinal motility disorders, constipation, regurgitation or irritable bowel syndrome. Preferred are the treatment or prevention of colic, particularly infantile colic or the treatment or prevention of necrotizing enterocolitis (NEC). Infantile colic and necrotizing enterocolitis (NEC) are described elsewhere herein.

In addition, for example through the positive effects on the health of the female or expectant mother conferred by the administration of probiotic microorganisms, the present invention also provides methods or uses which can prevent or reduce the risk of pre-term or premature birth and/or low birth weight of the neonate. In particular, the treatment or prevention of gingivitis or a urinary tract infection in a pregnant (or yet to be pregnant) female is likely to contribute to the prevention or reduction in risk of pre-term birth and/or low birth weight of the fetus/neonate.

In such aspects, it is generally envisaged that a probiotic microorganism would be chosen which was suitable for treating gingivitis or urinary tract infection (or other disease or condition) in the pregnant (or yet to be pregnant) female and this probiotic microorganism would not only have a positive effect on such diseases and conditions in the pregnant (or yet to be pregnant) female, but this beneficial microorganism would also be passed or transferred to the fetus in accordance with the methods or uses of the present invention and then in turn to the neonate and to the infant which developed from said fetus. Alternatively, a mixture of probiotic microorganisms can be used in the methods of the present invention, in which case a suitable microorganism can be selected and used for treating any condition the pregnant (or yet to be pregnant) female is suffering from, and a different (or further) microorganism can optionally be selected for the benefit of the fetus, neonate or infant. These microorganisms can then be administered together, for example at the same time, e.g. in a mixture, or separately. The microorganisms can also be administered at different times if desired.

The therapeutic uses of the present invention as described herein generally result in the reduction or alleviation of the relevant disease or condition, or symptoms thereof. Such reduction or alleviation of disease or conditions or symptoms thereof can be measured by any appropriate assay depending on the disease in question. The reduction or alleviation of disease, condition, or symptoms is preferably clinically significant. Preferably the reduction or alleviation of disease, condition or symptoms is statistically significant, preferably with a probability value of <0.05. Such reduction or alleviation of disease, condition or symptoms are generally determined compared to an appropriate control individual or population, for example a healthy mammal or an untreated or placebo treated mammal.

As will be clear from the disclosure elsewhere herein, the methods and uses of the prevent invention are suitable for prevention of diseases as well as treatment of diseases. Thus, prophylactic treatment is also encompassed by the invention. For example, it can be seen from the discussion herein that some of the diseases to be treated by the methods and uses of the prevent invention are not actively suffered by the fetus but only manifest symptoms in the neonate or infant. Thus, prevention of disease, for example in the neonate or infant is an important aspect. For this reason in the methods and uses of the present invention, treatment also includes prophylaxis or prevention where appropriate. Preferred aspects where prevention is envisaged are discussed herein.

As the methods of the invention allow the transfer or administration of a probiotic microorganism from the mother to the fetus, the passing of these probiotic microorganisms subsequently from the fetus to the neonate and then to the infant which develops from the fetus thereby means that the methods and uses of the present invention can be used to treat or prevent diseases in the neonates and infants as well as the fetus per se.

Thus, the present invention also provides combination therapies in which diseases or conditions can be treated or prevented in the mother and also in the fetus (and thereby the neonate or infant). Although the probiotics administered for said combination therapies are preferably the same probiotics, they can also be different.

Appropriate probiotic microorganisms for individual or combined treatments of the invention can readily be selected by a person skilled in the art. For example, appropriate probiotic microorganisms can be selected based on the disease or condition to be prevented or treated in the fetus, neonate or infant in accordance with the present invention. For example, preferred are probiotic microorganisms which are known for their capability of preventing (prophylactic treatment) or treating colic, e.g. infantile colic, or to minimize the risk for developing colic. Other preferred probiotic microorganisms are those which are known for their capability to have an anti-inflammatory effect (e.g. for their anti-inflammatory characteristics) for example in the preventing (prophylactic treatment) or treating of NEC, or to minimize the risk for developing NEC. Other preferred probiotic microorganisms are those which are known for their capability of reducing allergy or an immune response, for example in the preventing (prophylactic treatment) or treating or reducing allergy or other immune associated conditions, or to minimize the risk for developing allergy or other immune associated conditions. In addition, the use of probiotics to enhance and improve the general health of the mother can have a benefit on the fetus, for example by the prevention or reduced risk of premature birth and/or low birth weights.

The administration of probiotic microorganisms in accordance with the present invention can also be combined with other therapeutics, for example other therapeutics which are useful for the treatment of the same disease or condition, for example traditional pharmaceutical therapeutics can also be administered. Such additional agents can be administered together with the probiotic microorganism as described herein (e.g. as a composition), or can be administered separately. In addition, such additional agents can be administered at the same time point as the probiotic microorganism as described herein or at different time points. Suitable administration regimes and timings can readily be determined by the skilled person depending on the further agent in question.

Any appropriate probiotic microorganism, for example any probiotic bacteria, can be used in the methods or uses of the present invention, in particular any probiotic microorganism which would be useful for treating diseases or conditions or have any health benefit in an infant, neonate, or fetus can be used in the present invention and can be administered or transferred to the fetus via administration to the mother as described herein. Preferred bacteria are lactic acid bacteria, for example Lactobacillus or Bifidobacterium. Particularly preferred probiotic bacteria are Lactobacillus reuteri, in particular the strains Lactobacillus reuteri DSM 17938 or ATCC PTA 5289. The Lactobacillus reuteri DSM 17938 strain was deposited at the DSMZ-Deutsche Sammlung von Mikroorganismen and Zellkulturen GmbH (Mascheroder Weg 1b, D-38124 Braunschweig) on 30 Jan. 2006. The Lactobacillus reuteri strain PTA-5289 was deposited at the American Type Culture Collection (Manassas, Va. 20110-2209, USA) on 25 Jun. 2003. These strains are useful for the treatment or prevention of infantile colic, NEC or for preventing premature birth or pre-term birth and/or low birth weight. The DSM 17938 strain is particularly preferred for the treatment or prevention of infantile colic or NEC and the ATCC PTA 5289 strain is particularly preferred for preventing premature birth or pre-term birth and/or low birth weight.

Use of these two strains (Lactobacillus reuteri DSM 17938 and ATCC PTA 5289) in combination is particularly preferred in the methods and uses of the present invention. For example these strains can be administered as a mixture in an appropriate ratio and dosage, e.g. a 1:1 ratio.

Appropriate dosages of these microorganisms (and indeed any of the probiotic microorganisms to be used in the methods and uses of the invention) can be readily determined by a person skilled in the art. For example, a dosage, formulation and administration regime is chosen such that the probiotic microorganism administered to the female in accordance with the present invention can colonize the oral cavity of the female and can be transferred (or administered) to the fetus in an appropriate amount to colonize the fetus and to give rise to the desired effect, e.g. the desired therapeutic effects. Thus, preferably said dosage is a therapeutically effective dosage which is appropriate for the subject being treated, e.g. the fetus, neonate, infant or female. Exemplary regimes are provided in the Examples. For example, when the microorganism is administered as a chewing gum or lozenge then an exemplary dose of microorganism is 2×10⁸ cfu per gum or lozenge. Doses of 10⁶ to 10⁹ microorganisms may equally be used. An exemplary regime is that 2 gums or lozenges are administered each day, preferably after mealtimes and preferably morning and evening. If the formulation is a chewing gum then the gum can for example be chewed for approximately 10 minutes.

The invention will be further described with reference to the following non-limiting Examples:

### EXAMPLES

### Example 1

### Manufacturing of Chewing Gum Products Containing a Probiotic Strain

In this example, Lactobacillus reuteri DSM 17938 or ATCC 5289 (FJ1) is selected in order to add the strain to a chewing gum. The Lactobacillus reuteri strain is grown and lyophilized, using standard methods for growing Lactobacillus in the industry.

The steps of an example of a manufacturing process of chewing gum containing the selected strain follow, with it being understood that excipients, fillers, flavors, encapsulators, lubricants, anticaking agents, sweeteners and other components of chewing gum products as are known in the art, may be used without affecting the efficacy of the product:

1 Melting. Melt Softisan 154 (SASOL GMBH, Bad Homburg, Germany) in a vessel and heat it to 70° C. to assure complete disruption of the crystalline structure. Then cool it down to 52-55° C. (just above its hardening point).
2 Granulation. Transfer Lactobacillus reuteri freeze-dried powder to a Diosna high-shear mixer/granulator, or equivalent. Add slowly during approximately 1 minute the melted Softisan 154 to the Lactobacillus reuteri powder. No additional massing time is required. Use chopper during the addition.
3 Wet-sieving. Immediately after the granulation, pass the granules through a 1-mm sieving net by using a Tornado mill. The sieved granulate is packed in alupouches, made out of PVC-coated aluminium foil, sealed with a heat sealer to form a pouch, together with desiccant pouch, and stored refrigerated until mixing. The granulated batch is divided for two tablet batches.
4 Mixing. Mix all the ingredients in a mixer, to a homogenous blend.
5 Compression. Transfer the final blend to the hopper of a rotary tablet press and compress tablets with a total weight of 765 mg, in a Kilian compressor.
6 Bulk packaging. The chewing gums are packed in alu-bags together with a drying pouch of molecular sieve. The alu-pouch is put in a plastic bucket and stored in a cool place at least one week, before final package.

In-process controls, as is standard in the industry, are shown in the following Table 1.

TABLE 1

| IPC | Test | Limit | Method |
|---|---|---|---|
| 1 | Appearance | Clear, homogenous solution | Visually |
| 2 | Temperature | 52-55° C. | Thermometer |
| 3 | Appearance | Cream coloured with blue spots, | Visually |
| | | convex tablets plain on both sides. | |
| 4 | Uniformity | 765 mg ± 5% | Ph. Eur. |
| | of mass | | |

### Example 2

A test group of pregnant women is included in a study. One subset of the test group receives Lactobacillus reuteri strain ATCC PTA 5289, in the form of lypholized powder in standard gelatin capsules, whilst the other subset of the test group receives Lactobacillus reuteri DSM 17938 in the form of a chewing gum. The intake of the probiotics is for the last three weeks of the women's pregnancy. Lactobacillus reuteri strain ATCC PTA 5289 is in the form of gelatin capsules due to the wanted effect of the probiotic to take place in the mother's gut. Lactobacillus reuteri DSM 17938 in the form of a chewing gum, on the other hand is given orally to the mother to maximize the exposure time and location exposure in the oral cavity and thus the successful colonization in the oral cavity. After colonization in the mother's oral cavity, the probiotic bacteria can then spread via the blood to the placenta and then further to the infant gut.

Stool samples can be collected from each subject at day of birth (day 0). The samples (no less than 2 g) is then collected into a sterile container and placed immediately in a refrigerator. Within 24 hours, 20 ml (1:5, wt/vol) of 0.1% peptone water is added. The sample is homogenized and aliquots are dispensed into cryo-vials before freezing immediately at −70° C.

Placental samples can also be collected. As soon as the placenta is delivered it is placed in a sterile container and 1 cm³ cuboidal sections are circumferentially excised from separate areas of the placenta and the placental surfaces are then discarded. After collection the samples are placed on dry ice in sterile closed vials and stored at −80° C.

The stool and placenta samples are shipped, frozen on dry ice, to Biogaia AB laboratory for analysis of total Lactobacillus and the two L. reuteri strains PTA 5289 and DSM 17938. There, the stool samples will be thawed, diluted and plated on MRS-3 agar containing vancomycin (50 mg/l) for the L. reuteri and LBS agar plates (KEBOLAB AB, Lund, Sweden) for the total Lactobacillus count. MRS-3 is a modified MRS agar (KEBOLAB AB, Lund, Sweden) containing 2% sodium acetate (wt/vol). LBS agar is prepared as recommended by the manufacturer by adding 1.32 ml glacial acetic acid per liter. Agar plates will be incubated anaerobically using BBL Gas packs in anaerobic jars) at 37° C. for 48 hours. DNA from selected isolates from the study is analyzed by PCR.

DNA will also be isolated from the placental samples. Approximately 100-150 mg of frozen placental tissue are taken from each sterile sample vial under ongoing strict decontamination conditions, homogenized and heated, and then processed using PowerSoil DNA Isolation Kit (MO-BIO Laboratories). The DNA samples will be stored in kit solution C6 at −20° C. DNA will then be analyzed by PCR.

### Example 3

A pregnant mother is subject to treatment with probiotics in order to reduce the risk of having a child with a non-optimal gastrointestinal microbiome. The pregnant mother will take oral probiotics, preferably certain lactic acid bacteria, for example Lactobacillus reuteri DSM 17938 during her pregnancy, and thereby transfer them to the fetus via the blood and the placenta.

### Example 4

A woman planning to get pregnant is subject to treatment with probiotics in order to reduce the risk of having a child with a non-optimal gastrointestinal microbiome. The woman will take oral probiotics, preferably certain lactic acid bacteria, for example Lactobacillus reuteri DSM 17938 before and during her pregnancy, and thereby transfer them to the fetus via the blood and the placenta.

### Example 5

Lactobacillus reuteri Detection of Mice Placenta and Puppies.

### Experimental Procedures

Animals bred in germ free captivity were housed in our germ-free facility. All materials including food paper-based bedding, rodent chow, and water was sterilized either through autoclaving or chemical treatment and passed aseptically. Experimental procedures were carried out aseptically within isolators and biosafety cabinets.

Timed pregnancies were used. At 0.5 day post-copulation (identified by a mating plug), dams were inoculated with pure cultured bacteria (10̂6-10̂9 bacterial cells) by oral gavage. The mice were monitored for signs of septicemia looking for signs of distress and monitoring core temperatures.

Oral swabs and cage stool were collected at days 1.5, 7, 14 and 20. At day 20, mice were sacrificed and spleen, liver, placenta, and the fetuses were also aseptically collected. The number of pups between the groups will be assessed. One whole decapitated fetus and a whole placenta from each pregnancy were formalin fixed and paraffin embedded.

Bacterial colonization of maternal and neonatal organs were assessed by multiple methods. Stool and oral swabs assessed the colonization throughout pregnancy using a combination of 16S metagenomic sequencing and qPCR.

Reuterin test was performed to confirm growth of L. reuteri. In this example L. reuteri 4020 was used.

### Results

The results are shown in the Table 2 below. Four mice were orally administered with L. reuteri and five mice were used as germ free control. In the control group there were no detection of L. reuteri neither in the placenta nor the pups. In the test group L. reuteri was found both in the placenta and the pups in two of the mice. In the test group all the four mice which were orally administered with L. reuteri were shown to have oral colonization with L. reuteri.

Detection of L. Reuteri

| | Oral administration of L. Reuteri | | | | Germ free control | | | | |
|---|---|---|---|---|---|---|---|---|---|
| placenta | + | − | + | − | − | − | − | − | − |
| pups | + | − | + | − | − | − | − | − | − |

Table 2 shows the detection of L. reuteri (+ or −).

### Example 6

Pregnant women will be approached at the Sahlgrenska University Hospital or other hospitals or antenatal clinics within the Western Health Care Region of Sweden. Pregnant women who are planned for a vaginal birth will be asked for participation. Inclusion and exclusion criteria are listed below.

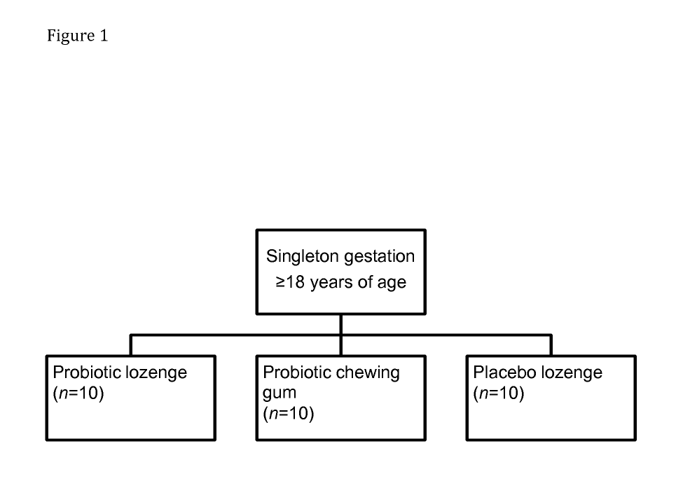

Enrollment into the study will be done in gestational week 28-36. In connection with enrollment, the women will be randomly selected to receive treatment with Lactobacillus Reuteri (provided by BioGaia Inc.) oral products (probiotic lozenges, probiotic chewing gums or placebo (lozenges)) (FIG. 1). Probiotic treatment means that the women consume supplements containing lactic acid bacteria. The probiotic products contain two Lactobacillus reuteri strains (1:1): L. Reuteri DSM17938 (isolated from breast milk of a Peruvian lady) and Lactobacillus reuteri ATCC PTA5289 (isolated from a Japanese woman's oral cavity). The concentration is 200 million cfu (2×10̂8) per lozenge or chewing gum. Other ingredients are hydrogenated palm oil, hydrogenated cotton seed oil, talc, menthol flavor, peppermint flavor, silicon dioxide, magnesium stearate, peppermint oil and sucralose. The chewing gum also contains a gum base. The ineffective lozenges have similar content but lacks the probiotic lactic acid bacteria, which means that they do not in any way effect the body. All products are vegetable and completely free of lactose and milk protein.

The participating women will take two lozenges/gums per day (morning and evening) after meals. The chewing gum should be chewed on for about 10 minutes. The exposure will last for the rest of the pregnancy. Treatment with lozenges will be double blinded, meaning that neither the women nor the doctor/midwife/researcher will know if the women have received lozenges containing probiotics or placebo until after completion of this study. This is made possible by all the lozenges having the same appearance, odor, weight and labeling. This does not apply for women being randomly assigned to chewing gum, since that is only available as a probiotic product.

Women participating in the study will be asked to refrain from the use of probiotic supplements, probiotic dairy products and other products and preparations containing probiotics during the study period. Women who are using any form of iron supplements will be asked to take these in an as far as possible interval from intake of trial preparations.

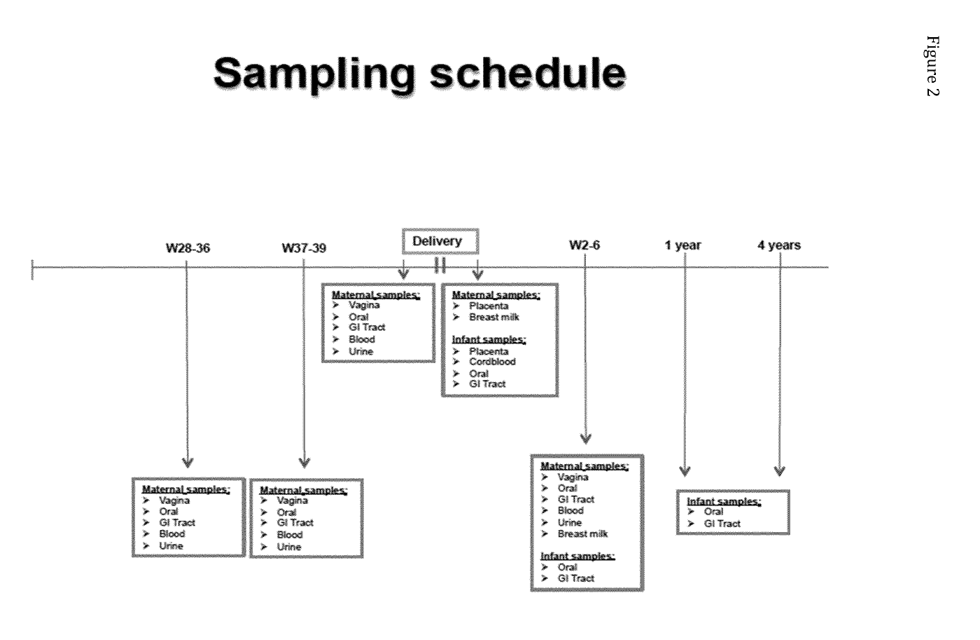

We will continue to enroll women until we have 10 completed subjects in each group. A completed subject is defined as a woman who has underwent all visit, where all samples have been collected, who has consumed the products she has been addressed and who has been followed up completely (FIG. 2).

The concentration of bacteria in the probiotic products will checked every 2-3 month during the study by BioGaia Inc.

The children born to mothers who are enrolled into this study will be followed up by sampling and medical records until the age of 18 years.

Collection of Clinical Specimens and Sampling Schedule (FIG. 2) In connection with enrolment at gestational week 28-36 (Visit 1), blood samples will be taken and a sample of the vagina, rectum/faeces, urine and oral cavity/saliva. In gestational weeks 37-39 (Visit 2), new samples will be collected from the same stations. After delivery (Visit 3), samples will be taken from the umbilical cord, placenta and breast milk, and from the mouth of the infant and the child's rectum/first stool (meconium). Women who deliver before Visit 2 will instead be sampled right before the delivery. Additional sampling will occur 2-6 weeks after delivery at a follow up visit (Visit 4).

If a subject has a body piercing within the area from which a specimen will be obtained (e.g., clitoris), the subject should remove any piercing jewelry or hardware on the day of specimen collection. Study staff should record the presence of a piercing in the applicable comments section of the visit documentation form.

Samples from the mouth of the infant and the child's rectum/faeces will also be collected at the age of 1 year (Visit 5) and 4 years (Visit 6). At these visits, the child's length and height will be measured and patient history will be collected.

Maternal and infant stool will either be collected at the visits when the mother brings a specimen or collected by mail.

TABLE 3

| Body Site | | Specimen | Visits | Acronym |
|---|---|---|---|---|
| Maternal | Vagina | Vaginal Introitus | 01, 02, 03, 04 | INTRO_01 |
| | | Posterior Fornix | 01, 02, 03, 04 | PFORN_01 |
| | Oral | Hard palate | 01, 02, 03, 04 | M_PAL_01 |
| | | Gingiva | | M_GNG_01 |
| | | Buccal | | M_BUC_01 |
| | GI Tract | Maternal Stool | 01, 02, 03, 04 | M_STOOL_01 |
| | Blood | Maternal Blood | 01, 02, 03, 04 | M_BLOOD_01 |
| | Urine | Maternal Urine | 01, 02, 03, 04 | URINE_01 |
| Placenta | Breast Milk | Breast Milk | 03, 04 | BMLK_01 |
| | Maternal side | Maternal placenta | 03 | MATPL_01 |
| | Fetal side | Fetal placenta | 03 | FETPL_01 |
| | Tissue Sample | Placenta Tissue | 03 | PLAC_01 |
| | Cord Blood | Cord Blood | 03 | CBLOOD_01 |
| Infant | Oral | Hard palate | 03, 04, 05, 06 | I_PAL_01 |
| | | Gingiva | | I_GNG_01 |
| | | Buccal | | I_BUC_01 |
| | GI Tract | Infant stool | 03, 04, 05, 06 | I_STOOL_01 |

Visit 1: 28 w 0 d-36 w 0 d
Visit 2: 37 w 0 d-39 w 0 d
Visit 3: Delivery
Visit 4: 2-6 weeks after delivery
Visit 5: Child visit, 1 years of age
Visit 6: Child visit, 4 years of age

### Biological Analysis

The main purpose of this study is to analyze microbial content and composition of bacteria in different compartments in the mother and the child. Both cultivation techniques for the specific bacteria, (e.g. Lactobacillus Reuteri) and metagenomic techniques (based on analysis of bacterial DNA) will be used to be able to study both cultivable and non-cultivable bacteria.

Different techniques will also be used for protein and cellular analyses in different samples. Examples of such techniques are proteomics, xMAP Luminex and FASC. We have used all of these techniques earlier. Further, ELISA will be used to study inflammatory markers such as calprotectin and myeloperoxidase (MPO).

Samples will be analyzed both in Sweden, Europe and USA for different types of analyzes.

### Medical Review

The medical records of the mother and fetus/child will be studied. Pregnancy, delivery, neonatal and child outcomes (up till 18 years of age) will be studied for all groups using maternal health care, women's clinic, children's clinic and child health care medical records and/or records from the National Board of Health and Welfare, Statistics Sweden and the Swedish Quality Registers (hold by Sveriges Kommuner och Landsting).

1-17. (canceled)

18. A method of delivering a probiotic microorganism to a fetus and/or neonate, comprising:
administering said probiotic microorganism to the oral cavity of a pregnant female in an amount of oral formulation suitable to colonize the oral cavity of said pregnant female, thereby delivering said probiotic microorganism to the fetus and/or neonate.

19. The method of claim 18, wherein delivery of the probiotic organism to the fetus and/or neonate treats or reduces the risk of developing infantile colic in the fetus and/or neonate.

20. The method of claim 18, wherein delivery of the probiotic organism to the fetus and/or neonate treats or reduces the risk of developing necrotizing enterocolitis (NEC) in the fetus and/or neonate.

21. The method of claim 18, wherein the colonization of the oral cavity of the pregnant female treats or reduces the risk of developing pre-term birth and/or reduces the risk of low birth weight.

22. The method of claim 18, wherein the colonization of the oral cavity of the pregnant female treats or reduces the risk of developing gingivitis or a urinary tract infection in the pregnant female.

23. The method of claim 18, wherein the probiotic microorganism is lactic acid bacteria.

24. The method of claim 23, wherein said lactic acid bacteria is a Lactobacillus and/or Bifidobacterium.

25. The method of claim 24, wherein said Lactobacillus bacteria is Lactobacillus reuteri, preferably Lactobacillus reuteri DSM 17938 and/or ATCC PTA 5289

26. The method of claim 18, wherein the oral formulation is a chewable tablet, chewing gum, lozenge and/or syrup.

27. The method of claim 18, wherein the formulation remains in the mouth of the pregnant female for a sufficient time to colonize the mouth of the pregnant female.

28. The method of claim 18, wherein the oral formulation comprises about 10⁶ cfu to about 10⁹ cfu of the probiotic microorganism.

29. The method of claim 18, wherein administering the probiotic microorganism to the oral cavity of the pregnant female occurs during a part of the pregnancy.

30. The method of claim 29, wherein the part of the pregnancy is the last few weeks of pregnancy.

31. The method of claim 29, wherein the pregnant female is a human and the part of the pregnancy is from week 28 to week 36 or is from week 28 to the end of the pregnancy.

32. A method for shifting the microbiome of a fetus and/or neonate, comprising:
administering a probiotic microorganism to the oral cavity of a pregnant female in an amount of oral formulation suitable to colonize the oral cavity of said pregnant female, wherein said probiotic microorganism is thereby delivered to the fetus and/or neonate, resulting in a shift in the microbiome of the fetus and/or neonate.

33. The method of claim 32, wherein the probiotic microorganism is lactic acid bacteria.

34. The method of claim 33, wherein said lactic acid bacteria is a Lactobacillus and/or Bifidobacterium.

35. The method of claim 34, wherein said Lactobacillus bacteria is Lactobacillus reuteri, preferably Lactobacillus reuteri DSM 17938 and/or ATCC PTA 5289.

36. The method of claim 32, wherein the oral formulation is a chewable tablet, chewing gum, lozenge and/or syrup.

37. A method of treating or reducing the risk of developing infantile colic or necrotizing enterocolitis (NEC) in a fetus and/or neonate, comprising
administering a probiotic microorganism to the oral cavity of a pregnant female in an amount of oral formulation suitable to colonize the oral cavity of said pregnant female, thereby delivering said probiotic microorganism to the fetus and/or neonate and treating or reducing the risk of developing infantile colic or NEC in the fetus and/or neonate.
